SEQUENCE LISTING (9 sequences)

SEQ ID NO: 1 - DNA, 117 bp, Saccharomyces cerevisiae
cagaaggaagaacgaaggaaggagcacagacttagattggtatatatacgcatatgtagtgttgaagaaacatgaaattgcccagtattcttaacccaactgcacagaacaaaaacc

SEQ ID NO: 2 - DNA, 13 bp, Artificial Sequence
  Description of Artificial Sequence Synthetic consensus sequence
  modified_base (6)..(8): a, c, g, or t
ggtcannntgacc

SEQ ID NO: 3 - DNA, 5835 bp, Aspergillus nidulans
gtcgactgcgtctgggtaagtgtcggccggtggctcgtttaggaggaatgacaacagggtcggtgtgcacgttcatgggctgggcgtctggtgctgtttcaagcgccatttcatcctgacgtagactcatatcgacagcggacggcgaaaccacaagttgttttggcgatgaagcagagctgaacaggaagctcctagggaccatcatgtttacttcttgaggccgcgaaaggtgtttttggaggcgtgttccaagcctgacgaggcactctgatcgagggtctgagcgagatacatggatgaatcgagaagggggcggcgaagagtccggtgtacttggtacggacgttcggcgttaggaaacgaaagaaagaaagagttgcggtgatgataaataatgtagtggtgagtagtagtgtagtgctgtagatccacccacaaccggtgtgttggcacgttaagcgtggaggcccgatgcgggagacccgttagagctggagtcggaaggacgcaggcacgcgacttgcaggtctcacggtcgggaagaacagccacgggaagggcctcgatgatcaaataaagagtctgaggctagctgacgccagcaacggatggtgctaaggaaagaagagagtgaaacagctgaaggcgattggaagaatggaagaatatggacggataagcatgatctcaatcagactcaaaagataaggaagatgcgggtgtgacggcatccgctggtggaccgtccgtttgggtctgttacttaccgtctgcacattacgctatccggtattagaattacaattagtaatgttatgcctttactatatggtttttgattggttcatatgaacccagtttctcttcggactacctagtctgtttctttgacagccagccatgacacataaatactggtactctcattacccttactccgagtatgtcacttccgtagtcttgcggaagagtcgatggagccaaaaggcctaaagcagcaaatgatcacaaagccacctaactagctagaaaatccatccccacgcaagaacattccatttctttttctggcccccgagcgaccgctactatccagccgaatgtttcaacagagtcagggataaaacttgcttcgtcgctgccttgctgatgcctgcgtgcccaacctcctcgttttctactagttttccactacactgtacatacaaattcgaggtattactaatccatgaggtggaatgagtgtcagggtagggaaggctcggcaacgagtcatctgaccggctctttcacagtaaacaaccagatctaactttactgacaaaagcaagcattgcagcatctcacaggacaactgaaaggaagacggtgctccgaagctgagctcagtagcactgtgacagagtgttcccaggtctgcatcttgttccagctgtcggcggaatctcggctttcttgcctccagtgaaaccggtgatcttcttggtatctctgtgcaagcatagaagcacatgctatcctctaccacagccatatacccagtatgagtgaaatacttgcgtcgtgtatagagtaagacaatagatataaagaactaccgctcatctgacagagggagaactgagttcaaaaagctgagatactgttctaacaaaaagagggcttagtcagctggcttatcagaccaaccgcaggaaatagttatgtttgaagcacatcactaatcagcctgtctccggcttcgctgagcagcttgctgctatctactccgtatcttgcacgcacactggcccaataatcttccttgtccttcagctgcccccagtctttggagtcggaaacgaatccaggccctccaccagattccaactgttcggattgtctctacccctccgtctccgaaagagcccgcctccacttgcgggtagcggggatttcgatattcgatcttggtgagagagctgtgagtactccaagatagacatagagtcaattatttggttgagggcatggccatcagcgcatagcctggtgctatgctctacgcgccgctatctttatcggttttattctaccgcgcatatctgacgcatacgccttttatatatcctagattgtaccattgcgagctacgataaccgtttcgatggtgtgcgcatcctgtcaagcctcagattcgcccaaattctcgtctccgtctcctcgtctcgttcgccatctatcacagggaaacacgccgagcgtcctcctccgataagcatgcgctgtcttggtctgtcgctgtcaccgaacatatctgtctgggcttgatttaccatgggggaaaagctcgacccggaactctcttccgacggacctcacacgaagtcgtcgtccaagggtcaagggaccagtacggacaatgctccagcgtcgaagcgacgttgtgtgagcacagcgtgcattgcttgccgcaggcggaaatcaaaggtaacaagtgccggccgctaggtttatatgcgttcaaagttctgacggtcttcaagtgtgatggaaatcttccaagttgcgccgcctgctcctccgtctatcatacaacctgtattgtgaccattcatcagctatcctcggaagatggctaatatatactaggcgtttatgatccaaactccgaccaccggcgaaaaggagtttacaagaaggatacggacacgttaagaactaagaactctaccttgttaactttgattcaggcgctgctcaactatgaggaggaagatgccttcgacctggtccgccaaatccggtcctgtgataacctggaagacgtagcgcaatctctcgtaaaccaggaaaagaagtcgtctggttggctgtcaaatgcggtaatacatgaagagaacgatatcgcgcagacggatcaattcgagtctgaattggctggaaagatgagcaacctcgtgctagacggatcccgcaagtttattggcgggacgtcaaacctcatcttcctaccacccggatcggaactcaacgagttcaagcctggcttggctactaacggcgatctggagggatccgtcacccggtggacgacagttactgatgatcagcagcttattagccatctcttgacaatgtacttttcatggcattatcctttcttcaccactctgtccaaggaactcttctaccgtgattactctcgtggagtgccgagccagtattgctcttccttgcttgttaacacgatgctggccttggggtgtcacttcagctcctggccaggggcccgtgaagatccagacaactctgctaccgccggtgatcactttttcaaagaagcgaagagattgatccttgacaacgacgagcttgtcaactcaaagctttgcaccgtgcaagctctggctcttatgtccgtacgagaggccggatgtggccgtgaagggaaaggctgggtttatagcggaatgagtttccgcatggcatttgacttgggcttgaatcttgagtcttcgagcttacgagatctcagcgaagaagagatagatgcaagacgcatcactttttggggttgtttcctcttcgacaagtatggtgctgtcaaaacttcacacagataaatgctaattgatctagatgttggtctaattatcttggacggcagcctcagttcacgacagcgaacacaagtgtttccgcagtggatattctacccaacgaagaatcaacattatggtctccttacagtgatatggggcctagtcgggagtatgcacaaccttcgcggacaagggcagtcgcagaccagatttcccaattgtgtaagatcagtggagatctagtggtgtttttctacgaccttgctcccaaagaaaagccatcgtccaagcaactggagctaaagaagctaagtgagattcatactcgcttagaagcgtggaagaaaggcttgccgaaggaactcgagccgcgcgaaggccagcttccgcaggcgctactcatgcagtaagttagtctaggaactgaaagctagcattgtacttacaccattacagcatgttctaccaacttctcctcattcacctttaccgccccttcctcaaatacacaaagtcgacctccccgcttcctcagcatgtctctccccgcaagctatgcactcaggcggcagctgctatatcgaagttactacggctttataaacgcacatacgggtttaagcagatctgcaatatcgccgtttatattgcgcatacagccttgacaatccaccttctcaatctacccgaaaagaatgcacagagagatgtaatacacggcctgagacatctcgaagagatgggagagtcttggctttgtgctcgccggacgcttcgtattctcgacatatctgccagtaagtggcaggtacaactaccgagggaggccgttatcgtcttcgaacaaacacatgctcgatggggctcgtggggcccttgggaccaagcagcatcgccttcaacaacctcggattcaccgccatccgtcagctcccagtccgtggttgccaccacggatctatcccaacctgtatcccagtctgcgggcaatcagcctgccaatccttctatgggtacgtctccgaacctaacacagcccgtagcctctcaatattcttccacgccttctggacctgtctctgtatctgcaatgcgtgctgttcaacgatctttcagtgctcaacttgcacacaacgaagcccgacaacctgaacctacgtatctaagaccggtctctaccagctatggaccagttccgtcaactcagtctgcacaagagcagtggtacagcccaactgaggcacaattccgcgcatttactgctgctcacagtatgcctacaacatcagcccaatctccgttgaccacctttgatacaccagaaaatctggttgaggaaagtcaggactggtggtctcgcgacgttaatgcactacaactcggggccgaggactggactcagaattggaataatggtctccccaccacatcggccgactggcgctatgtggacaatgtccccaacatcccttctacatctgcgcccgatgccgattacaagcctccacaaccacctcccaacatggcaagaccgaatcaatatccaactgaccctgttgcgaacgtgaacagcaaccaaaccaacatgattttccccggcagttttcagcgatagcaggcctattcaccgctgcggataattgctcttcatgggcttggtcttgacgacgaatagactcggtgtgcatgactttacgatgtgattaagataattcattgacctaggcgactcttcattaaatatatgtatcatgccttctccatattttaacacttctctcgaataggcagttggtggactcgaatctcaggcgttggttctatatgtgcgtgatatatgccttacaccttatactttatactccgtattcgtgttcatttttcctcatttaccatatatacttgattttctagagagtcaatggccttccctttccgtttcagtgctcacccctcgccgctttttgctcactgagtagacctcacgcgctgagcacgctctggacgaagctcgccgctcgtcgctgagcaccctttcggacagctagaataaagggacttgaatatctcatagtgaatggcggaggctactcagaagacggaagatgggcaccttgctcccatctgccaagtcaatgcctacattgtccaccgtggtcttctcaggtagctgcacaacaggatcttcatcattctgatatgggcccagtccatgcgaatcgagccctcctacatgggcttgtagtag

SEQ ID NO: 4 - DNA, 94 bp, Aspergillus nidulans
agggaaacacgccgagcgtcctcctccgataagcatgcgctgtcttggtctgtcgctgtcaccgaacatatctgtctgggcttgatttaccatg

SEQ ID NO: 5 - DNA, 287 bp, Aspergillus nidulans
tgctatgctctacgcgccgctatctttatcggttttattctaccgcgcatatctgacgcatacgccttttatatatcctagattgtaccattgcgagctacgataaccgtttcgatggtgtgcgcatcctgtcaagcctcagattcgcccaaattctcgtctccgtctcctcgtctcgttcgccatctatcacagggaaacacgccgagcgtcctcctccgataagcatgcgctgtcttggtctgtcgctgtcaccgaacatatctgtctgggcttgatttaccatg

SEQ ID NO: 6 - DNA, 382 bp, Aspergillus nidulans
cgatattcgatcttggtgagagagctgtgagtactccaagatagacatagagtcaattatttggttgagggcatggccatcagcgcatagcctggtgctatgctctacgcgccgctatctttatcggttttattctaccgcgcatatctgacgcatacgccttttatatatcctagattgtaccattgcgagctacgataaccgtttcgatggtgtgcgcatcctgtcaagcctcagattcgcccaaattctcgtctccgtctcctcgtctcgttcgccatctatcacagggaaacacgccgagcgtcctcctccgataagcatgcgctgtcttggtctgtcgctgtcaccgaacatatctgtctgggcttgatttaccatg

SEQ ID NO: 7 - DNA, 568 bp, Artificial Sequence
  Description of Artificial Sequence Synthetic construct nucleotide sequence
tccatgcagttggacgatagcttccatggtcacagtgaccggtcacagtgaccggtcacagtgacccagaaggaagaacgaaggaaggagcacagacttagattggtatatatacgcatatgtagtgttgaagaaacatgaaattgcccagtattcttaacccaactgcacagaacaaaaacctgcagatattcgatcttggtgagagagctgtgagtactccaagatagacatagagtcaattatttggttgagggcatggccatcagcgcatagcctggtgctatgctctacgcgccgctatctttatcggttttattctaccgcgcatatctgacgcatacgccttttatatatcctagattgtaccattgcgagctacgataaccgtttcgatggtgtgcgcatcctgtcaagcctcagattcgcccaaattctcgtctccgtctcctcgtctcgttcgccatctatcacagggaaacacgccgagcgtcctcctccgataagcatgcgctgtcttggtctgtcgctgtcaccgaacatatctgtctgggcttgatttaccatg

SEQ ID NO: 8 - DNA, 473 bp, Artificial Sequence
  Description of Artificial Sequence Synthetic construct nucleotide sequence
tccatgcagttggacgatagcttccatggtcacagtgaccggtcacagtgaccggtcacagtgacccagaaggaagaacgaaggaaggagcacagacttagattggtatatatacgcatatgtagtgttgaagaaacatgaaattgcccagtattcttaacccaactgcacagaacaaaaacctgcagctatgctctacgcgccgctatctttatcggttttattctaccgcgcatatctgacgcatacgccttttatatatcctagattgtaccattgcgagctacgataaccgtttcgatggtgtgcgcatcctgtcaagcctcagattcgcccaaattctcgtctccgtctcctcgtctcgttcgccatctatcacagggaaacacgccgagcgtcctcctccgataagcatgcgctgtcttggtctgtcgctgtcaccgaacatatctgtctgggcttgatttaccatg

SEQ ID NO: 9 - DNA, 280 bp, Artificial Sequence
  Description of Artificial Sequence Synthetic construct nucleotide sequence
tccatgcagttggacgatagcttccatggtcacagtgaccggtcacagtgaccggtcacagtgacccagaaggaagaacgaaggaaggagcacagacttagattggtatatatacgcatatgtagtgttgaagaaacatgaaattgcccagtattcttaacccaactgcacagaacaaaaacctgcagggaaacacgccgagcgtcctcctccgataagcatgcgctgtcttggtctgtcgctgtcaccgaacatatctgtctgggcttgatttaccatg

(19) United States
(12) Patent Application Publication
(10) Pub. No.: US 2007/0077642 A1
(43) Pub. Date: Apr. 5, 2007

(54) Steroid receptor modulation of gene expression

(76) Inventors: Joseph Strauss, Vienna (AT); Robert Pachlinger, Vienna (AT)

Correspondence Address: MERCK AND CO., INC, P O BOX 2000, RAHWAY, NJ, 07065-0907, US

(21) Appl. No.: 10/541,901

(22) PCT Filed: Dec. 23, 2003
(86) PCT No.: PCT/US03/41357

§ 371 Date: Jun. 16, 2006

Related U.S. Application Data
(60) Provisional application No. 60/436,506, filed on Dec. 23, 2002.

(51) Int. Cl. C12N 15/74; C12N 1/18

(52) U.S. Cl. 435/254.2; 435/483; 435/484

(57) ABSTRACT

The present invention provides a novel steroid inducible expression system in a non-mammalian host cell (e.g., fungal) that is independent of metabolic and developmental regulation. The human estrogen receptor gene expressed in Aspergillus, under a constitutive promoter, was shown to be functional. A construct containing a promoter from Aspergillus, synthetic sequence containing the estrogen receptor biding sites (EREs) and a reporter gene was expressed in response to a hormone derivative inducer. In the absence of the inducer, the promoter is silent and depending on the type of construct and inducer concentration the expression level can be modulated from moderate to very strong.

### FIELD OF THE INVENTION

The present invention generally relates to the field of inducible expression systems. In particular, the present invention relates to modulation or regulation of gene expression.

### BACKGROUND OF THE INVENTION

Steroid hormones diffuse across plasma membranes, bind to and activate targeted receptors. Upon activation, steroid receptors bind to and regulate the transcription of DNA. This physiological method of gene regulation has been used as a blueprint for a number of gene expression systems.

It is well-known that a steroid hormone may induce transcription of a subset of genes in specific cell types. Transcriptional activation of steroid responsive genes (through interaction of chromatin with hormone receptor/hormone complex) is effected through binding of the complex to enhancer sequences associated with the genes.

A number of steroid hormone and thyroid hormone responsive transcriptional control units have been identified. These include the mouse mammary tumor virus 5′-long terminal repeat (MTV LTR), responsive to glucocorticoid, aldosterone and androgen hormones; the transcriptional control units for mammalian growth hormone genes, responsive to glucocorticoids, estrogens, and thyroid hormones; the transcriptional control units for mammalian prolactin genes and progesterone receptor genes, responsive to estrogens; the transcriptional control units for avian ovalbumin genes, responsive to progesterones; and mammalian metallothionein gene transcriptional control units, responsive to glucocorticoids.

The initiation of transcription occurs when an active transcription complex assembles at a promoter, which in turn dictates where transcription will start. A promoter region contains several domains, which are necessary for full function of the promoter. The first of these domains lies immediately upstream of the structural gene and forms the core promoter region containing consensus sequences, usually about 70 base pairs (bp) immediately upstream of the coding region. The core promoter region contains the characteristic CAAT and TATA boxes plus surrounding sequences, and represents a transcription initiation sequence that defines the transcription start point for the structural gene. The precise length of the core promoter regions is variable but it is well recognizable by the skilled worker. This region is normally present, in some variation, in all promoters.

The core promoter region is insufficient to provide full promoter activity. Regulatory sequences, usually upstream from the core, constitute the remainder of the promoter. The regulatory sequences determine expression level, the spatial and temporal pattern of expression and, for an important subset of promoters, inducible expression. These are responsive to intra- and extracellular signals.

The steroid receptors activate or repress transcription when bound to upstream elements (“hormone response elements” or “HREs”). These HREs are specific enhancer sequences and are recognized by specific hormone receptors, thus assuring that a distinct response is triggered by distinct hormones. For example, ligands such as 17β-estradiol or an estrogen derivative (e.g., diethylstilbestrol or zearalenone) bind to the estrogen receptor's ligand binding site. This binding event triggers a conformational change of the receptor and the migration of the ligand-receptor complex from the cytoplasm to the nucleus, where the receptor recognizes these specific HREs, binds to the HRE nucleic acid sequence, interacts, either positively or negatively with the transcriptional machinery, thereby affecting gene expression.

Each steroid receptor harbors a DNA binding domain that binds to the HRE sites on the DNA. These binding domains have been well characterized (Evans, R. M., Science 240, 889-895 (1988); Giguere, V. et al., Cell 46, 645-652 (1986)). Steroid receptors additionally share regions of structural and/or functional homology. The organization of each of these regions is divided into distinct domains, with each domain being conserved in all members of the hormone gene superfamily. These domains correspond to the variable N-terminal region (domain A/B), zinc finger DNA binding region (domain C), hinge region (domain D), the C-terminal ligand binding region (domain E), and the variable C-terminus (domain F). See Evans, Science, 240, 889-895 (1988).

The N-terminal domain is highly variable in size and sequence and poorly conserved among the members of the superfamily. This particular domain functions in the modulation of transcription activation (Bocquel et al., Nucl. Acid Res., 17, 2581-2595 (1989); Tora et al, Cell, 59, 477-487 (1989)).

The DNA-binding domain (DBD) targets the receptor to specific HREs within the transcription control unit of specific target genes on the chromatin (Martinez and Wahi, Nuclear Hormone Receptors, Acad. Press, 125-153 (1991)).

The ligand binding domain (LBD) is essential for recognizing and binding to the receptor's cognate ligand. The ligand binding domain also possesses a transcriptional activation function. Altogether, the LBD aids in determining the specificity and selectivity of the hormone response of the receptor. LBDs are well-known to vary considerably in homology between the individual members of the nuclear hormone receptor superfamily (Evans, Science, 240, 889-895 (1988); P. J. Fuller, FASEB J., 5, 3092-3099 (1991); Mangelsdorf et al., Cell, Vol. 83, 835-839 (1995)).

The functions present in the N-terminal region, LBD and DBD are independent from one another. It has been shown that these domains can be exchanged between nuclear receptors (Green et al., Nature, Vol. 325, 75-78 (1987)). The result of such exchanges is the “chimeric nuclear hormone receptor.”

There are several naturally occurring regulatory systems that are well characterized and exist in bacteria. Such systems use the interactions between DNA binding proteins and their target DNA sequences to induce, enhance, attenuate, or repress gene expression. A gene activation system that has been well characterized is the nitrate assimilation system of Aspergillus nidulans. Both pathway induction and nitrogen metabolite repression regulate nitrogen metabolism in Aspergillus nidulans. The pathway specific transcriptional activator NirA mediates pathway induction (for review, see Scazzocchio and Arst, Regulation of nitrate assimilation in Aspergillus nidulans; Molecular and Genetic Aspects of Nitrate Assimilation, Wray, J. L., and Kinghorn, J. R. (eds.), Oxford: Oxford Science Publications, pp. 299-313 (1989)). It was previously shown that the genes encoding for nitrate reductase (niaD) and nitrite reductase (niiA) are co-regulated in response to the availability of nitrate (Punt, P. J. et al., Mol. Cell. Biol. 15, 5688-5699 (1995)). Binding of NirA was shown to depend on intracellular nitrate and a functional AreA protein, a member of the so-called GATA factor family of transcription factors (Narendja, F. et al., Molecular Microbiology, 44(2), 573-583 (2002); Wilson and Arst Microbiol. Mol. Biol. Rev., 62, 586-596 (1998); and Scazzocchio, C., Curr. Opin. Microbiol. 3, 126-131 (2000), and references cited therein). GATA factors exist in all eukaryotic kingdoms from man to molds and regulate diversification processes such as vertebrate cell differentiation (Patient R. K. et. al., Curr. Opin. Genet. Dev., 12(4), 416-422 (2002)) pathogenicity factors in phytopathogenic fungi (Voisard et al., Mol. Cell. Biol. 13, 7091-7100 (1993)), or primary metabolism in fungi and yeasts (Gomez et al., Mol Microbiol., 50(1), 277-89 (2003), Magasanik and Kaiser, 2002, Gene, 290:1-18). Examples are: AreA in A. nidulans, the homologues in N. crassa, Nit2 and yeast S. cerevisiae Da180, Gln3, Nil1 and Nil2. All genes mentioned are involved in the regulation of nitrogen acquisition. There are additional examples such as regulators of light signal transduction in Neurospora, of siderophore biosynthesis in several fungi; there are also plant GATA factors, but the function is still unknown.

Several of these naturally occurring regulatory systems have been exploited in yeast and other microbial systems to construct heterologous gene expression systems that are dependent upon metabolites such as lactose to generate an activating or repressive gene expression response. While these systems provide a certain level of control over the expression of a target sequence, they are governed by molecules that ultimately affect the host cell's metabolism and exhibit undesired pleiotropic effects on host cell genes. The use of heavy metals or carbon sources as inducers of specific physiological activities and gene expression place an additional burden on the host cell as it tries to metabolize and recover from extraordinary high levels of the inducer.

Retroviral vectors have been used to introduce tetracycline inducible systems into mammalian cell hosts to drive the expression of genes of interest. However, the yeast and microbial strains are not amenable to retroviral vector delivery methods.

Many of the metabolite induction-based systems in mammalian and microbial cells are limited due to the relatively slow and inefficient activation of gene expression by inducer.

Typical biopharmaceutical processes employ constitutive promoters where cell growth and production are coupled. Over the course the fermentation process, it is intuitive that the largest possible number of cells produce therapeutic proteins in the shortest possible period of time. Thus, in order to produce a high titer of quality proteins, a decoupling of the growth phase from the production phase would be advantageous. Proteins produced under constitutive promoters can be subject to degradation, covalent modification and interfere with metabolism of the host cell as detailed below. Therefore, the expression of genes in complex genetic environments, such as yeast and other microbial strains, would greatly benefit from systems that would allow stringent control of the expression of individual genes in order to generate proteins of interest that are manipulated in vivo, both spatially and temporally.

Generally, important aspects of temporal and spatial stringent gene expression control for the production of therapeutic proteins include use in defined minimal media, inexpensive ingredients, inducers in low concentrations (for example, storage and handling of inducer is usually largely free of hazardous manipulations—e.g. methanol is removed). The induction process is initiated quickly (optimally within minutes after induction), the inducer should be chemically stable and biologically active after a prolonged time of incubation in the medium. Additionally, the inducer may be antagonizable with a chemical compound and should be easily removable from the fermentation broth. Inducible gene expression may be used predominantly for the production of heterologous proteins. These proteins may be therapeutic.

Inducible gene expression allows growing cells to grow to a sufficient density. By subsequent induction of gene expression, the production phase is initiated. The production rate of a desired protein product can, in addition, be directly modulated by the concentration of inducer, added during fermentation. In the absence of inducer, the gene of interest is not expressed, thereby avoiding any potential toxic effects of heterologous protein expression until expression of the product is induced.

During expression, changes due to the inducer compound necessary to drive the expression may result in toxicity and limited uptake of the carbon source. For example, the addition of methanol using the Aox promoter in methylotrophic yeast, such as Pichia sp., may limit carbon availability. Moreover, specific media composition necessary to allow expression usually hinders efficient biomass accumulation. For example, alc-promoter expression systems usually function only in low glucose conditions. These expression systems rely on strong constitutive promoters such as GAPDH or inducible systems derived from metabolic activities such as the alc-system. What is needed, therefore, is an inducible expression system that regulates gene expression in the presence of an inexpensive inducer, which is independent of metabolic and development regulation.

### SUMMARY OF THE INVENTION

The specificity of a steroid receptor, such as the estrogen receptor, for its target hormone responsive sequence, as well as the high affinity of steroids, e.g., estrogen and estrogen-like molecules, for their cognate receptor and the well-studied chemical and physiological properties of steroid receptors and ligands, constitute a basis for a highly efficient regulated inducible expression system in non-mammalian host cells, especially in yeast and other microbial strains. Further, hormones, e.g., steroid hormones such as diethylstilboestrol (DES), can easily be antagonized or withdrawn from the medium by biochemical methods. Accordingly, a steroid inducible promoter expression system in a filamentous fungus is provided. In one embodiment, a system comprises:

(i) a nuclear hormone steroid receptor or a first nucleic acid which, upon expression in a host cell produces an encoded nuclear hormone steroid receptor;

(ii) a second nucleic acid comprising (a) a target nucleotide sequence to be transcribed operatively linked to (b) a promoter core and (c) at least one hormone steroid response element; and (d) a stuffer fragment juxtaposed between (b) and (c); whereby expression of the target nucleotide sequence from the promoter core is induced in the presence of nuclear hormone steroid.

### BRIEF DESCRIPTION OF THE DRAWINGS

FIG. 1 depicts a plasmid map of phERpyr4.

FIG. 2 depicts a plasmid map of pRM2124.

FIG. 3 depicts a plasmid map of pRM2119.

FIG. 4 depicts a plasmid map of pRM2085.

FIGS. 5A, 5B and 5C depict construction of the three consecutive estrogen response elements genetically linked to URA3, nirA promoter, which in turn is linked to a reporter protein.

FIG. 6 depicts growth activity of transformed A. nidulans argB2, riboA1, pyrG, pyroA4 on Xgal plates at different ligand concentrations.

FIG. 7 shows the results of an assay for β-Gal activity in liquid culture containing Xgal.

FIG. 8 depicts an assay of β-gal activity in A. nidulans transformed with pRM2085, pRM2119 and pRM2124.

FIG. 9 depicts an assay of β-gal activity in A. nidulans transformed with pERE reporter constructs in comparison to alcA expression.

FIG. 10 depicts a calibration curve of protein concentration.

FIG. 11 depicts an assay of β-gal activity in different metabolites.

FIG. 12 depicts an assay of β-gal activity in different metabolites.

FIG. 13 depicts plasmids containing the JUNK element modulating protein expression.

### DETAILED DESCRIPTION OF THE INVENTION

In one embodiment the present invention provides a regulated gene expression cassette comprising a first promoter operatively linked to a regulator protein, such as a nuclear hormone, e.g., a steroid protein, and a regulatable (e.g., an inducible) promoter operatively linked to a target nucleic acid molecule (e.g., gene), the inducible promoter being activated by the regulator protein in the presence of an effective exogenous inducer, whereby administration of the inducer causes expression of the target gene and removal or addition of antagonist of the inducer stops expression. In another embodiment, the regulator protein is expressed from a nucleic acid construct that is not physically linked to the nucleic acid molecule comprising the promoter region to which the regulator protein binds. It is to be understood, however, that the gene encoding the regulator protein and the target gene or sequence to be expressed may reside in the same nucleic acid construct. In this case, the presence of a suitable inducer, the regulator protein produced by the first cassette will activate the expression of the target gene by stimulating transcription from the inducible promoter in the second cassette. Alternatively, one or both constructs may be stably integrated into the host cell's genome.

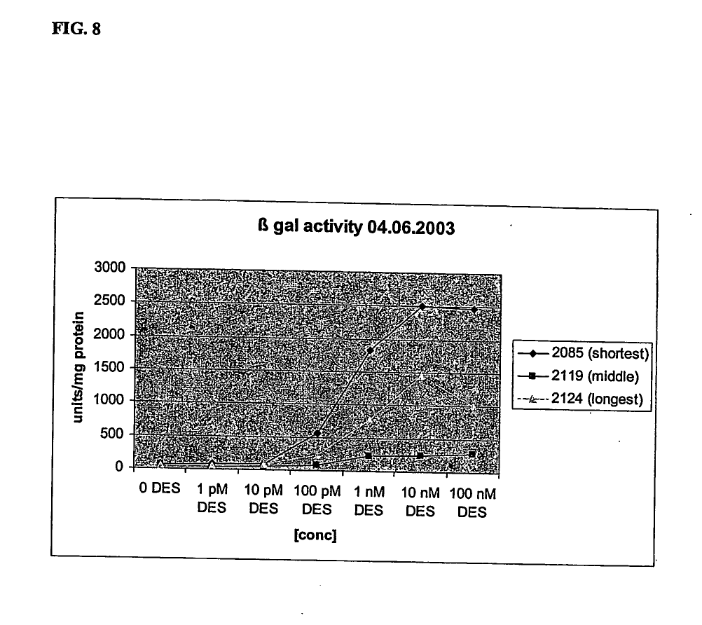

In another embodiment, steroid receptor binding sites are introduced into the promoter of the nirA gene. The core inducible promoter is operatively linked to a target gene or sequence. It is to be understood that such a sequence may be cDNA or DNA. The “core” promoter is defined by the characteristic CAAT and TATA boxes plus surrounding sequences, and represents a transcription initiation sequence that defines the transcription start point for the structural gene. Immediately upstream of the core promoter region is a “stuffer-fragment”, between about 75 and 250 bp, preferably between about 100 and 200, and more preferably between about 100 and 150 bp in length, which provides optimum spacing between the transcriptional machinery bound to the core promoter and the HRE bound steroid receptor further upstream. This spacing and the nucleotide composition of the spacer is critical for regulation (i.e., activation or repression) of expression of the target nucleic acid, e.g., a gene of interest. A promoter fragment that contains the EREs in a different spacing may be not functional (FIG. 8).

There are several well-established modes of gene activation that a transcriptional activator protein may use to exert influence. For example, an activator may exert influence via a shifting of histones or alteration of chromatin structure thereby allowing an efficient start to transcription, via direct contact with the transcriptional machinery already assembled at the core promoter, via recruitment of the necessary factors for transcription, via providing a bend(s) or twist(s) in the DNA to allow proper contact between transcription factors and the polymerase, or via providing a bend(s) or twist(s) in the DNA to allow efficient binding of the necessary components for transcription. It is to be understood that any of these modes may represent the mechanism for which a gene is activated and expressed herein.

In one embodiment, the host cell used in the systems and methods of the invention is a non-mammalian host cell, and preferably a fungal (e.g., yeast) or bacterial cell. Yeast, fungal, and/or bacterial cell systems are used because they lack endogenous nuclear receptors and other receptor co-regulatory proteins that are found in more complex mammalian cell lines. They are also advantageous because they are easily amendable to genetic and biochemical screening and selection techniques, which allows the skilled worker to vary parameters and select out of huge populations the rare cells that behave in a desired manner. Yeast and filamentous fungi have both been successfully used for the production of recombinant proteins, both intracellular and secreted (Cereghino, J. L. and J. M. Cregg, FEMS Microbiology Reviews 24(1), 45-66 (2000); Harkki, A., et al., Bio-Technology 7(6), 596 (1989); Berka, R. M., et al., Abstr. Papers Amer. Chem. Soc. 203, 121-BIOT (1992); Svetina, M., et al., J. Biotechnol. 76(2-3), 245-251 (1992)).

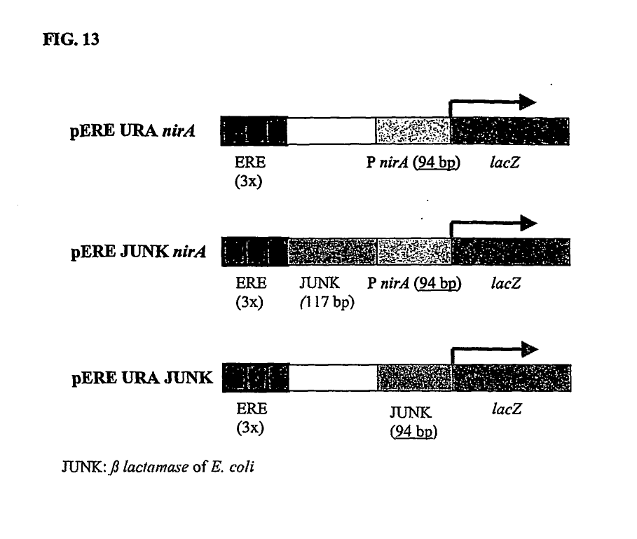

In another embodiment, the steroid inducible promoter expression system disclosed herein is easily transferred to unicellular yeasts, such as, but not limited to Pichia sp. (e.g., P. pastoris), Saccharomyces sp. (e.g., S. cerevisiae) and Kluyveromyces sp. (e.g., K. lactis) by a person of ordinary skilled in the art. In one embodiment, the estrogen inducible promoter elements are engineered in a P. pastoris strain YJN165, which is a uracil auxotroph strain lacking the P. pastoris URA5 gene (Nett et al., Yeast, 20(15), 1279-90 (2003)). A set of plasmids harboring P. pastoris URA5, or any other suitable selection marker, and a library of inducible promoter cassettes, such as pERE-URA-nirA, pERE-JUNK-nirA and pERE-URA-JUNK (FIG. 13), or any other combination of such elements described herein, or known to a person of skill in the art, make up the inducible promoter expression systems of the present invention. Preferably, the inducible elements are cloned upstream of a gene of interest and the whole DNA element comprising targeting elements for homologous integration (if homologous integration is chosen), such as the HIS3 and HIS4 genes (Cosano et al., Yeast, 14(9), 861-7 (1998)), a suitable selection marker, inducible promoter elements and the gene of interest is transformed and more preferably integrated into the host cells genome by methods known to a skilled artisan, such as employing chemically competent cells (Hanahan et al., Methods Enzymol., 204, 63-113 (1991)).

In another embodiment, a similar expression plasmid harboring a selection marker and the gene encoding for the steroid receptor (e.g., estrogen) driven by a strong GAPDH promoter, or any other suitable promoter known to the artisan skilled in the art, and, preferably, sequences derived from the genome of the host cell for targeting to an integration locus, such as the HIS3 and HIS4 genes (Cosano et al., Yeast, 14(9), 861-7 (1998)) or any other suitable locus, can be transformed into the cell of interest, and integrated into the host genome by homologous recombination.

The inducible system of the present invention provides several advantages; namely, the ability to turn on and off transcription completely in non-mammalian hosts. The inducible system is adapted to use inexpensive inducers such as DES in low concentrations (10 pM-10 nM). Additionally, the inducible system has no metabolic switches during induction. Other advantages including the ability to optimize fermentation conditions, use of inexpensive media, ability to secrete heterologous proteins, posttranslational modification e.g., glycosylation, lack of inclusion bodies, genetic stability of introduced genes make protein production in filamentous fungi optimal hosts.

A nucleic acid of the invention (e.g., one which encodes a nuclear hormone receptor or one which comprises the cognate hormone responsive element operatively linked to the core promoter and target gene construct) may be integrated into the genome (chromosome) of the host cell. Integration may be promoted by inclusion of sequences that promote recombination with the genome, in accordance with well-established techniques. Alternatively, DNA sequences, replicating independently from the genome (chromosome), may be used to express the gene(s) of interest (hormone receptor and/or operatively linked target gene) from an extrachromosomal locus (e.g., a replicating plasmid).

Introduction of a nucleic acid construct is referred to herein, without limitation, as “transformation”. Transformation may be accomplished by any available technique. Techniques include calcium chloride or lithium acetate transformation, electroporation, phage transfection, direct injection and the like. The skilled worker will be able to select an appropriate method for introducing a nucleic acid into a host cell depending on the host cell selected.

Marker genes such as genes complementing auxotrophes, antibiotic resistance or sensitive genes may be used in identifying clones containing the nucleic constructs of interest. For instance, the presence of a marker is useful in the subsequent selection of transformants; e.g. in yeast the URA3, HIS4, SUC2, G418, BLA, or SH BLE (and in Aspergillus: the argB, pyrG, riboB, niaD, hygB genes) genes may be used. In addition, well-known genetic manipulation techniques may be used to increase or attenuate steroid sensitivity (e.g., Multiple Drug Resistance gene deletions, ABC transporter deletions, or knockouts, using random or directed chemical or enzymatic mutagenesis techniques in conjunction with appropriate screens or selections for host cells having desired phenotypes or traits).

The host cell may be co-transformed with the two nucleic acid molecules (i.e., two different vectors), the first nucleic acid encoding a steroid receptor and the second nucleic acid vector harboring the nucleic acid to be transcribed operatively linked to a promoter core, a stuffer fragment linked to the 5′ end of the promoter core and at least one steroid response element linked to the 5′ end of the stuffer fragment, respectively. Either, or both, of the two nucleic acid molecules may be integrated into the host chromosome.

In a preferred embodiment of the invention, the target nucleic acid sequence encodes a glycoprotein that is to be secreted from the host cell. As such, this depends upon the presence of a cellular target sequence or signal peptide. In eukaryotic cells, proteins can be targeted for secretion, to the cell membrane, or to one of the many internal organelles. Intracellular proteins can be targeted to the cytoplasm, to the nucleus or to special organelles such as the mitochondrion or the chloroplast. For example, one may use genetic engineering techniques to add a signal peptide to the N-terminus of cytoplasmic proteins, such as globin, which naturally have no such sequences. This results in the engineered protein entering the endoplasmic reticulum (ER) and having the signal sequence cleaved off. The protein is then targeted through the ER and Golgi apparatus to the cell surface.

Metabolite Independent Regulatable Expression System

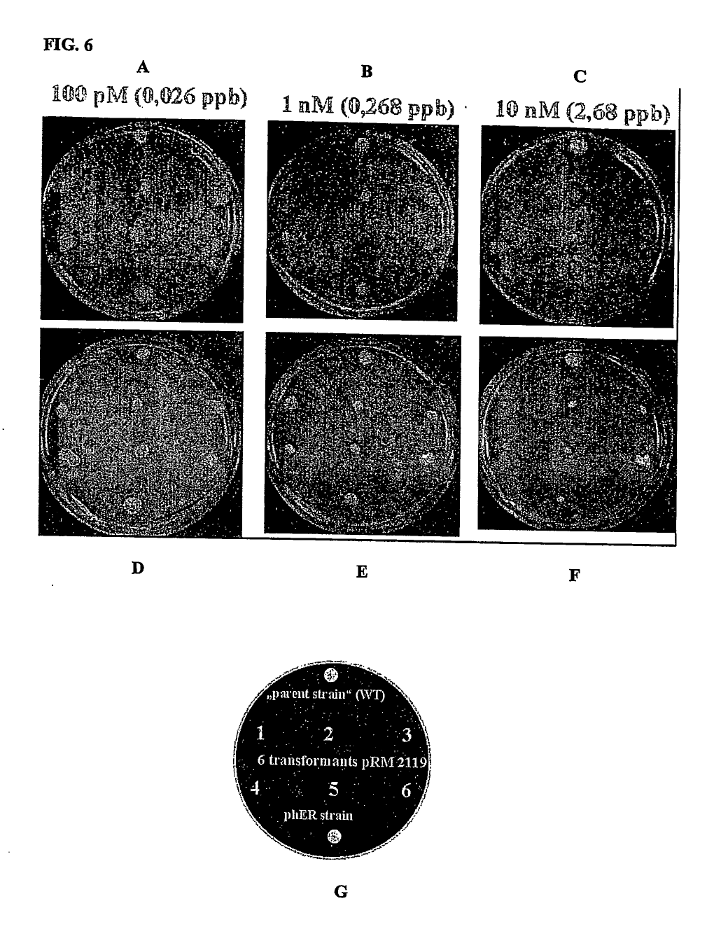

As stated above, a major advantage of using a nuclear hormone, e.g., a steroid inducible system in yeast, fungi, and bacteria is that steroid receptor responsive inducer molecules may be utilized without ever affecting the metabolic pathways of the host fungal, yeast, or bacterial organism. The independence from physiological signals of the cell in heterologous protein expression has been elusive in the art. The present invention provides a novel steroid regulatable expression system in a non-mammalian host cell (e.g., fungal) that is independent of metabolic and development regulation comprising a combination of negative and positive elements in the presence of hER. The present invention also provides methods for regulating gene expression in filamentous fungi and yeast that is independent of carbon and nitrogen regulation. The estrogen inducible expression system as exemplified herein is independent of metabolic and developmental regulation (FIG. 6). Intermediate expression levels, as well as fine-tuning, are achieved by modular combinations of elements with different inducer concentrations.

Inducer Molecules

Inducer compounds are readily added to the media or culture in which the host cells are proliferating. The inducer compounds are taken up by the cells, or passively diffuse through the cell membrane, and bind to their cognate receptor, thereby inducing a conformational change in the receptor and allowing binding to cognate hormone response element(s). Methods are well established that allow for the withdrawal of inducer from the media (i.e. use of column chromatography), or the withdrawal of inducer activity from the media (i.e. by the addition of inducer antagonizing chemical compounds). This represents an improvement over previous inducible expression systems and methods in lower eukaryotic and bacterial host cells.

Examples of ligands that will bind to estrogen receptors and either induce or repress gene expression from the target nucleic acid include: 17β-estradiol, diethylstilbestrol (DES), zearalenone (ZON), fixed ring 4-hydroxytamoxifen, non-steroidal stilbene analogs, tamoxifen, any of selective estrogen receptor modulators (SERMS), 4-1-(p-hydroxyphenyl)-2-phenylethyl]phenoxyacetic acid, raloxifene, estrogen, ICI164384 (pure ER antagonist), and ICI 182,780. Other well-known ligands (and their cognate receptors) include: cortisol (CORT receptor), androgen (Androgen receptor), progesterone (Progesterone receptor), aldosterone (Mineralcorticoid receptor), non-steroid hormones including: triiodothyronine (T3 receptor), dihydroxyvitamin D3 (D3 receptor), and two classes of retinoid (all-trans retinoic acid and 9-cis retinoic acid) receptors (RARs and RXRs, respectively). It is well recognized that differing concentration of estrogen receptor substrate, as well as addition of an estrogen receptor co-factor such as RIP140 (either synthesized in vivo via gene expression or added exogenously), will influence the level of transcription activation or repression.

Plant and fungal derived steroid like compounds may also be used to induce steroid receptor mediated gene expression. Examples of plant derived estrogen compounds include classes of chemical structures including flavones, isoflavones, flavanones, coumarins, chalcones and mycoestrogens. Phytoestrogens and plant lignans, abundantly found in soy products, have powerful estrogenic properties. 8-prenylnaringenin (8-PN) is a phytoestrogen present in hops and beer, whose functionality is inhibited by ICI 182,780, and mimics the effects of 17β-estradiol. The major isoflavin from licorice root extract, glabridin, exhibits varying degrees of estrogen receptor agonist in vivo and in vitro. Glagrene and isoliquiritigenin (2′, 4′, 4-three hydroxy chalcone) are known to bind to estrogen receptors and exhibit agonistic activity (both derived from licorice root extract). Several mycotoxins expressed in phytopathogenic Fusarium strains, such as Deoxynivalenol (DON) or zearalenone are known mimics of estrogens and activate hER. Thus, any of the foregoing plant, or fungal, derived estrogen-like compound examples may be used to induce estrogen receptor mediated gene expression.

Stuffer Fragment

The stuffer fragment provides the optimal spacing between the “activated” hormone (e.g., steroid) receptor and the core promoter-bound polymerase. This optimum spacing can provide for a proper shifting of histones or alteration of chromatin structure thereby allowing an efficient start to transcription, may provide for direct contact with the transcriptional machinery already assembled at the core promoter, may provide for accessible nucleic acid for the recruitment of necessary factors for transcription, may provide a proper bend(s) or twist(s) in the DNA to allow proper contact between transcription factors and the polymerase, or may provide a bend(s) or twist(s) in the DNA to allow efficient binding of the necessary components for transcription. It is to be understood that any of these modes may represent the mechanism for which the stuffer fragment functions, thereby allowing gene expression or repression.

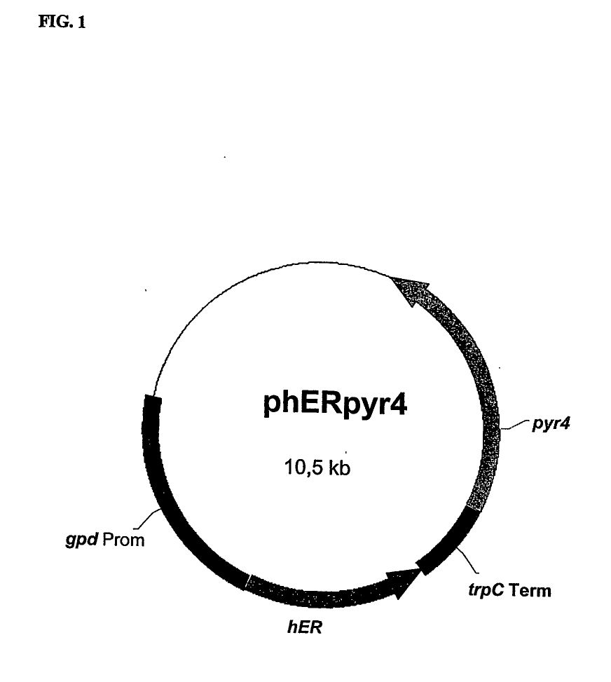

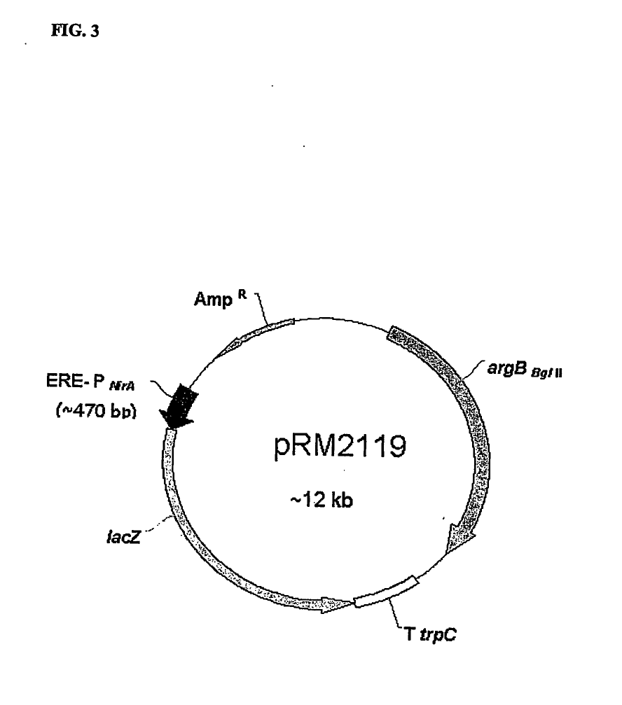

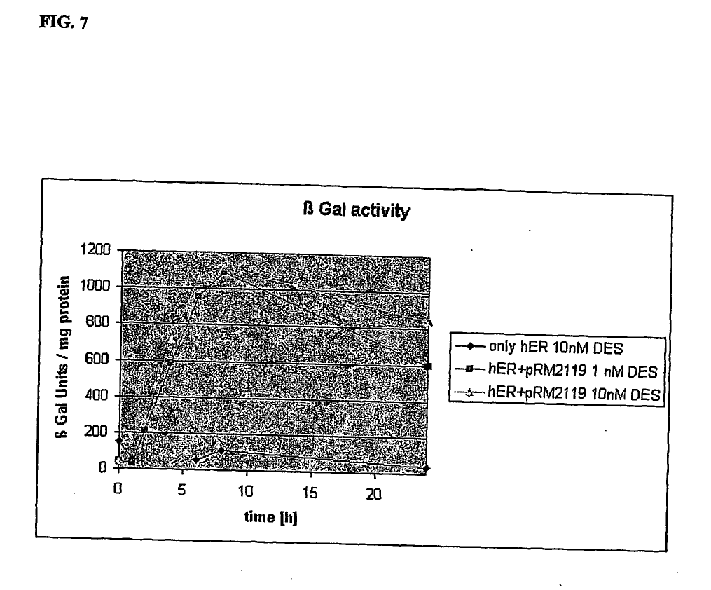

Preferred stuffer fragments will be between about 75 and about 250 nucleotides in length. More preferably, preferred stuffer fragments will be between about 100 and about 200 nucleotides in length, and still more preferably, between about 100 and about 150 nucleotides in length. One of ordinary skill in the art will recognize that random stuffer fragment nucleic acids may be screened for desired length and gene expression activity using well established methods in the art. Preferred stuffer sequences include the URA 3 nucleic acid fragment (SEQ ID NO:1) and a 97 bp fragment (including the ATG) of the nirA A. nidulans promoter as shown in FIG. 3. The URA 3 stuffer fragment together with selected nirA promoter DNA fragments provides desired gene expression in A. nidulans induced with DES (FIG. 1) and (FIG. 7). Certain combinations of URA3 promoter fragment and a NirA gene promoter fragment, in the absence of a stuffer region, results in the inability of the ER to activate expression of the reporter gene following hormone stimulation. For example, a direct fusion of the ura3 fragment with a nirA promoter fragment containing sequences rich in CT and an additional TATA sequence provide a construct that is not inducible by DES (See Example 2). Stuffer fragments may be selected and improved on the basis of whether or not they provide the particular desired gene expression properties.

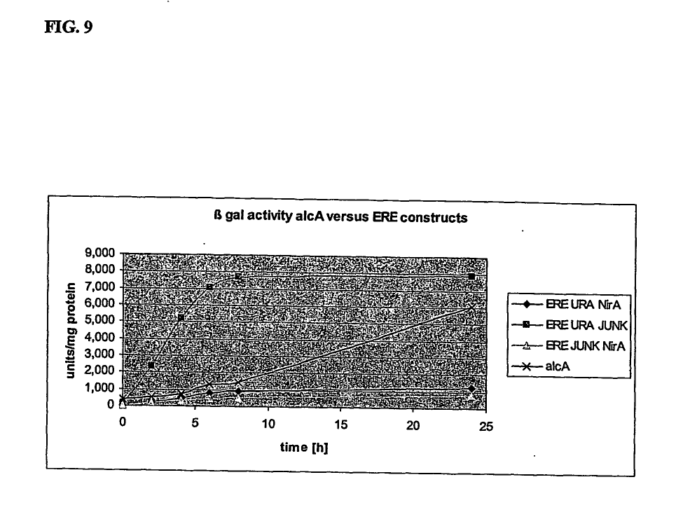

In a more preferred embodiment, the combination of an estrogen response element genetically linked to the URA3 nucleic acid fragment genetically linked to a JUNK element, initiate transcription in levels equivalent to that of alcA (FIG. 9). JUNK elements include, without limitation, any nucleotide combination, however, β-lactamase of E. coli is preferable. What is surprising about the combination is that the relative position of the JUNK element influences the level of induction dramatically (FIG. 9). The inducible expression system with a JUNK element can be screened and modified to modulate expression to a desired level. To date, the influence of promoter set-ups for the activity of an ERE in combination with other motifs has not been determined in yeast or filamentous fingi. The present invention, therefore, addresses the need for an inducible expression system that can be modulated with relative ease and independence of any metabolites.

Steroid Response Elements

Hormone response elements (HREs) are short cis-acting sequences (about 20 base pairs in size) that are required for hormonal activation of gene expression. HREs may be operably linked to coding sequences that are otherwise hormone non-responsive. Such a linkage provides for a gene that is now hormone responsive HREs are distinguished from other enhancer sequences based upon their dependency upon the presence or absence of hormone or ligand.

Binding of an agonistic or antagonistic ligand to a steroid receptor most often results in the dimerization of the receptor. For example, estrogen receptors α and β can homodimerize, and less frequently, heterodimerize. The ligand bound receptor recognizes and binds to sequences in regulatory regions of target genes (steroid response elements). Receptors bound to their cognate response elements may induce a bend in the DNA that facilitates the interaction of key transcriptional components. Once bound, the receptors may function as general transcription factors, co-activators, repressors, and proteins that regulate chromatin remodeling, signal for the activation or repression of target gene expression.

In one embodiment, the mode of achieving gene repression using a modified activator protein is via mutating the activation region and constructing a promoter in which the EREs are overlapping with naturally occurring enhancer sequences. Thus, the activation of mutated human estrogen receptor (hER) gene expression by estrogen, hER protein will bind to EREs and thereby compete with the natural activator.

An example of a steroid receptor that is well characterized and functions in this manner is the estrogen receptor. Activated estrogen receptors bind to DNA sequences (estrogen response elements, or EREs) with high affinity. Estrogen receptors bind to palindromic repeats in a dimeric head to head arrangement. The minimal ERE consensus sequence is defined in SEQ ID NO: 2 (5′-GGTCAnnnTGACC-3′, wherein n is any nucleotide).

As used herein, engineered HREs refer to HREs that have been recombinantly produced using genetic engineering techniques such as nucleotide substitution, deletion, etc. Additionally, HREs of the present invention may be synthesized in vitro using techniques well established in the art (such as automated nucleotide synthesis).

It is to be understood that the nucleic constructs encoding the steroid receptor of interest are not limited to encoding naturally occurring steroid receptors. It is well-known in the art that chimeric receptors may be constructed and utilized in gene expression systems.

Steroid Receptors

Nucleic acids encoding the hormone, e.g., steroid receptor of interest may be constructed using techniques familiar to one of ordinary skill in the art. Based upon the modular nature of the steroid receptor super family, as described above, novel nuclear hormone receptors have been constructed and described previously (see, for example, EP 0798 378 A2). Methods to prepare genetic constructs expressing receptor proteins in vivo are well known in the art (see, for example, Sambrook et al., Molecular Cloning: a Laboratory manual, Cold Spring Harbor Laboratory Press, Cold Spring Harbor, 1989; Ausubel et al., Current Protocols in Molecular Biology, John Wiley and Sons, (1994 and Supps. to 2002)). Multiple copies of steroid receptor encoding nucleic acid constructs may be integrated for optimal expression. Furthermore, the steroid receptor may be transcribed constitutively or in a regulated manner. Given the state of the art, regulated expression of the receptor may be desired and methods for generating such regulation are well known. Chimeric receptors comprising the LBD and/or the DBD of different receptors may be produced by chemical linkage, but most preferably the coupling is accomplished at the DNA level with standard molecular biological methods by fusing the nucleic acid sequences encoding the necessary steroid receptor domains. As noted above, the steroid receptors may function as general transcription factors, co-activators, repressors, and proteins that regulate chromatin remodeling, signal for the activation or repression of target gene expression.

Core Promoter

The core promoter may be representative of any naturally occurring core promoter that is normally “off” in the absence of any transactivating proteins. In other words, a substantial level of gene expression is initiated only in the presence of “activating proteins” in combination with RNA polymerase. An example of such a promoter is the nirA promoter, which contains sequences onto which general transcription factors and polymerase protein subunits assemble. It is well-known in the art that the TATA sequence in the core promoter (located approximately 25 nucleotides upstream of the transcription start site) is recognized by TATA binding protein, and this initial binding triggers the assembly process of the other transcription binding factors and RNA polymerase required for transcription. Given the nature of the promoter, however, high-level transcription in the presently constructed setting is properly initiated when the appropriate upstream (5′) sequences are bound by activated steroid hormone receptors. Once bound, these sequences, in combination with the stuffer fragment provide for the desired gene expression activation.

The core promoter may vary in length depending on the sequence required to properly bind the full complement of general transcription factors and polymerase.

A preferred core promoter is the nirA promoter (SEQ ID NO:3). The 94 bp fragment of the nirA promoter is also preferred (SEQ ID NO:4). A 287 bp fragment of the nirA promoter containing additional CT-rich sequences and a TATA sequence may also be incorporated into the genetic construct but this construct is devoid of activation function triggered by activated hER and the ERE. (SEQ ID NO:5). Still, another embodiment utilizes a 382 bp fragment of the full length promoter nirA (SEQ ID NO:6). In addition, random stuffer sequences may be selected on the basis of gene expression activation and/or repression. Methods are well developed in the art for the screening or selection of sequences that modulate, attenuate, repress, or activate gene expression (for example, using reporter gene technology).

Target Gene Encoding Protein of Interest

The target gene nucleic acid sequence is prepared using information and references contained herein and techniques known in the art (for example, see Sambrook, Fritsch and Maniatis, Molecular Cloning, A Laboratory Manual, Cold Spring Harbor Laboratory Press (1989); Ausubel et al., Current Protocols in Molecular Biology, John Wiley and Sons, (1994)). These techniques include (i) the use of polymerase chain reaction (PCR) to amplify sequences of interest (for example, from genomic sources), (ii) chemical synthesis, or (iii) the preparation of cDNA sequences. It is to be understood that the nucleic acid sequence encoding the protein of interest may be generated and used in any suitable way known to those of skill in the art, including identifying restriction enzyme recognition sites 5′ and 3′ to the sequence to be expressed, cutting out the sequence to be expressed from its original source or vector, and operably linking the sequence to a suitable promoter in the present system. Another recombination approach is to amplify sequence of interest with suitable PCR primers. Modifications to the relevant sequence may be incorporated using, for example, site directed mutagenesis.

In a preferred embodiment, recombinant proteins expressed from target nucleotide sequences of the invention, e.g., by steroid induction in engineered lower eukaryotic hosts, may be further engineered to be “human-like” glycoproteins (i.e., glycoproteins which are similar, if not substantially identical, to their human counterparts) using methods disclosed, e.g., in WO 02/00879, the specification of which is incorporated herein by reference. The unicellular and multicellular fungi disclosed therein are amenable for use in the presently disclosed regulatable gene expression system. The lower eukaryotes, which ordinarily produce high-mannose containing N-glycans, including unicellular and multicellular fungi may be modified to produce N-glycans such as Man₅GlcNAc₂ or other structures along human glycosylation pathways. Such fungi include, without limitation, Aspergillus nidulans, Aspergillus niger, Aspergillus oryzae, A. awamori, A. chrysogenum, A. saitoi, A. tubigensis, Trichoderma reesei T. viridae, T. harzianum, Trichoderma sp., Chrysosporium lucknowense, Fusarium sp., Fusarium gramineum, Fusarium venenatum, Mucor sp., Ashbya gossipii, Penicillium sp., and Neurospora crassa. Similarly, methylotrophic yeast is used, including for example, Pichia pastoris, Pichia finlandica, Pichia trehalophila, Pichia koclamae, Pichia membranaefaciens, Pichia minuta (Ogataea minuta, Pichia lindneri), Pichia opuntiae, Pichia thermotolerans, Pichia salictaria, Pichia guercuum, Pichia pijperi, Pichia stiptis, Pichia methanolica, Pichia sp., Hansenula polymorpha, Hansenula sp., Kluyveromyces sp., Kluyveromyces lactis, Candida albicans, Candida sp., and Torulopsis sp. P. pastoris is the host of a more preferred embodiment.

Constitutive Promoter

The promoter driving expression of the steroid receptor is preferably a constitutive promoter. Well-known examples of constitutive promoters exist. Examples of constitutive promoters include, for example, GAPDH (TDH3), ADH1, the enolase promoter, or the glyceraldehyde-6-phosphate dehydrogenase (gpdA) promoter. A constitutive promoter is defined herein as a promoter that requires no inducer and is sufficiently active to direct expression of an amount of steroid receptor protein effective to activate or repress the target gene of interest.

The presently disclosed invention will be further described below by way of the following non-limiting Examples and appended figures.

### EXAMPLE 1

### Nucleic Acid Constructs and Transformation of Strain

The following is a general strategy for constructing plasmids to be used for the transformation of the strain of interest.

Construction of Plasmid phERpyr4 (FIG. 1)

A 1.8 kb EcoRI fragment, containing the hER, of the plasmid Yep90-HEGO (Pierrat et al., 1992) was cloned into the NcoI site of a P_{gpd}-T_{trpc} construct (originated from pAN52-1) into a pBKSII+ backbone (Stratagene, La Jolla, Calif., USA) harbors the constitutive promoter gpdA, from Aspergillus nidulans (Acc. Z32524), which drives expression of the human estrogen receptor 1 (Acc. NM 000125). Termination sequences harbor the trpC terminator from Aspergillus nidulans (Acc X02390). The marker gene is a pyr4 (orotidine-5′-phosphate decarboxylase) from Trichoderma harzianum (Acc. U05192). The resulting P_{gpd}-hER-T_{trpc} cassette was cloned as a SpeI/ClaI fragment into a plasmid that contains the pyr4 gene of Trichoderma reseei as a selection marker resulting in plasmid phERpyr4.

General Structure of lacZ Reporter Constructs

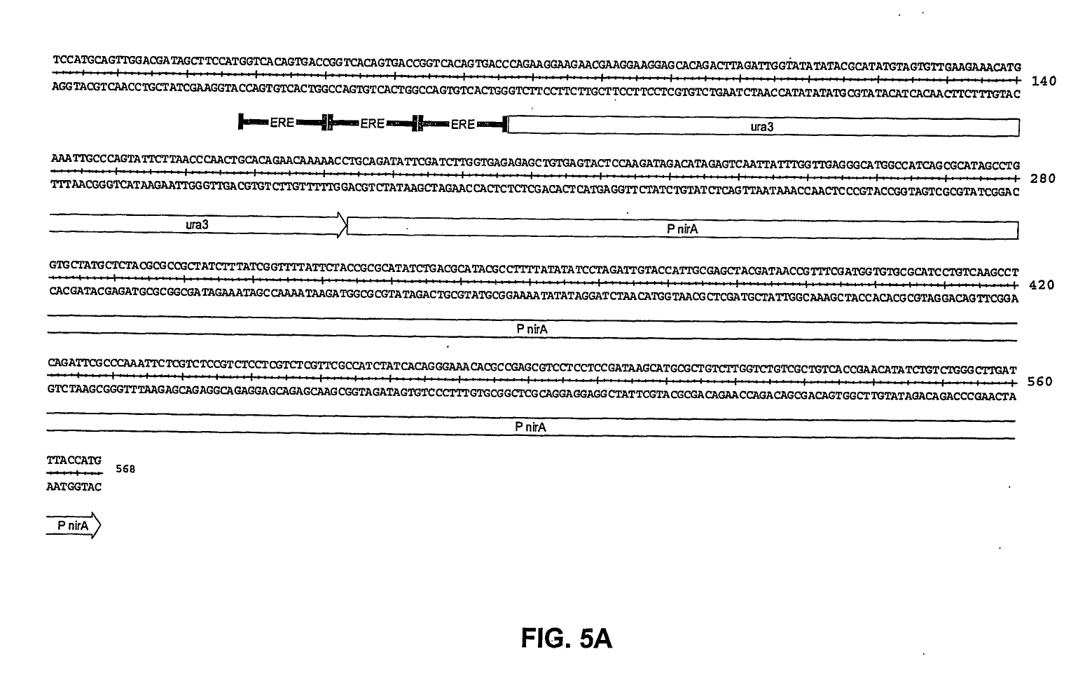

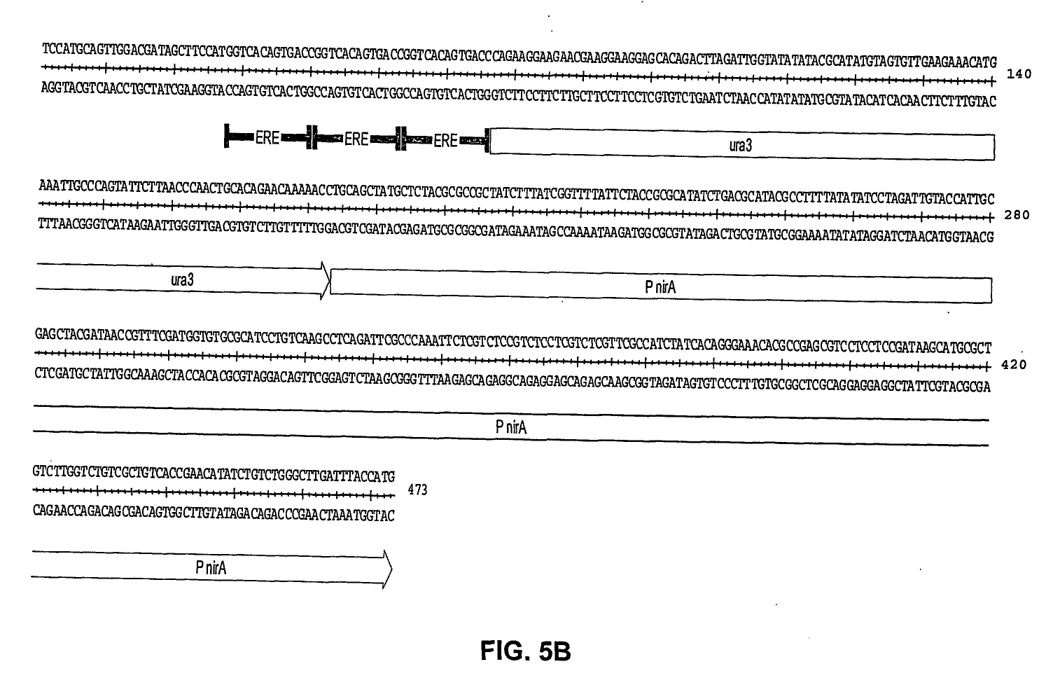

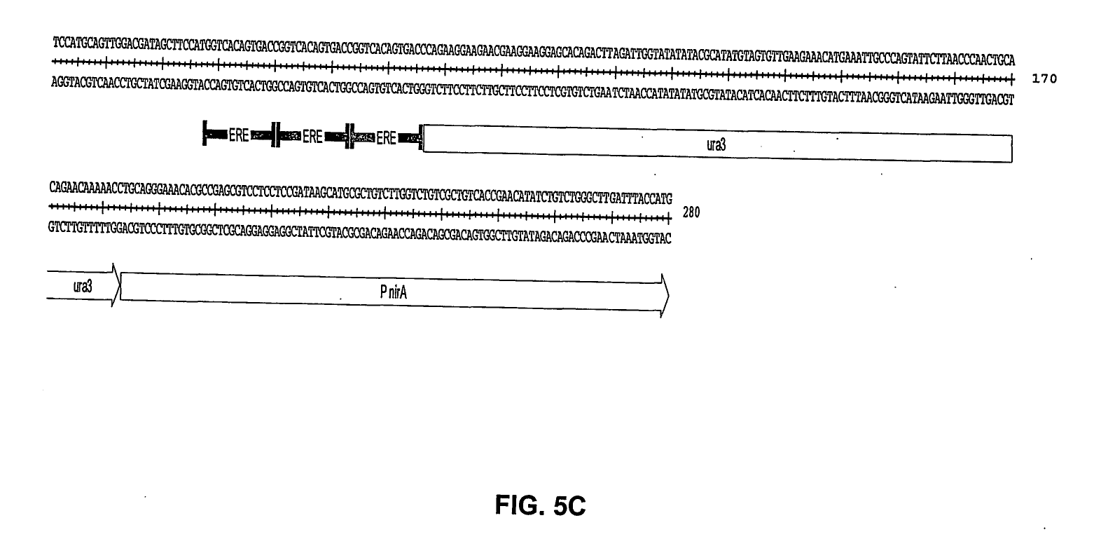

The cis acting element: three consecutive estrogen response elements (EREs) are genetically linked to 5′ end of URA3 stuffer fragment. The 3′ end of the stuffer fragment is genetically linked to the 5′ end of part of the nirA promoter of A. nidulans including ATG (FIGS. 5A, 5B and 5C).

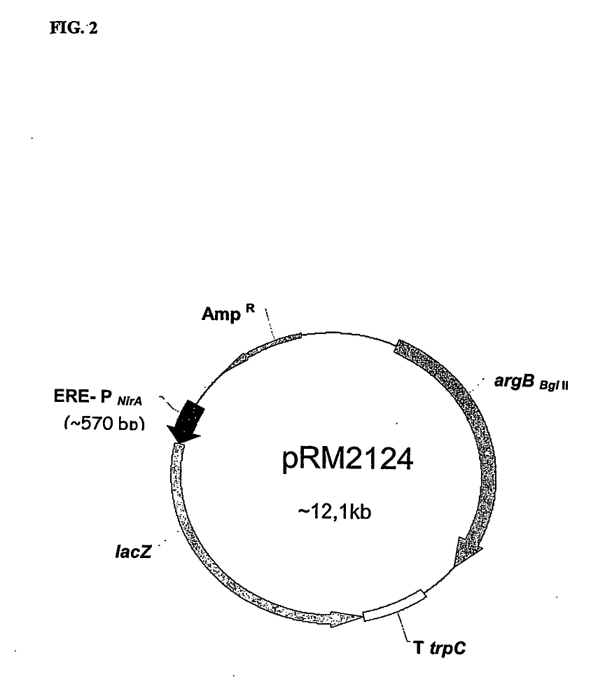

Construction of Plasmid pRM 2124 (FIG. 2)

3xERE sequence followed by a 117 bp fragment of the URA3 promoter of S. cerevisiae (Acc. M12926, bases 89-205) (Klein-Hitpass et al., 1992) and a 387 bp fragment of the nirA promoter of A. nidulans (Acc. M68900) was amplified by using PCR and was cloned as a BamHI/BglII fragment into the BamHI site of pAN923-42_{BglII} (Punt et al., Gene 93, 101-109 (1990)). The structural gene is the reporter lacZ from E. coli (Acc. V00296). The marker gene is argB from A. nidulans (Acc. AB020737). Terminator sequences include the trpC terminator from Aspergillus nidulans (Acc. X012390).

Construction of Plasmid pRM2119 (FIG. 3)

3xERE sequence followed by a 117 bp fragment of the URA3 promoter of S. cerevisiae (Acc. M12926, bases 89-205) (Klein-Hitpass et al., 1992) and a 292 bp fragment of the nirA promoter of A. nidulans (Acc. M68900) was amplified by using PCR and was cloned as a BamHI/BglII fragment into the BamHI site of pAN923-42_{BglII}. The structural gene is the reporter lacZ from E. coli (Acc. V00296). The marker gene is argB form A. nidulans (Acc. AB020737). Terminator sequences include the trpC terminator from Aspergillus nidulans (Acc. X02390). No reporter gene expression is observed in yeast cells harboring this construct when inducer is exogenously supplied.

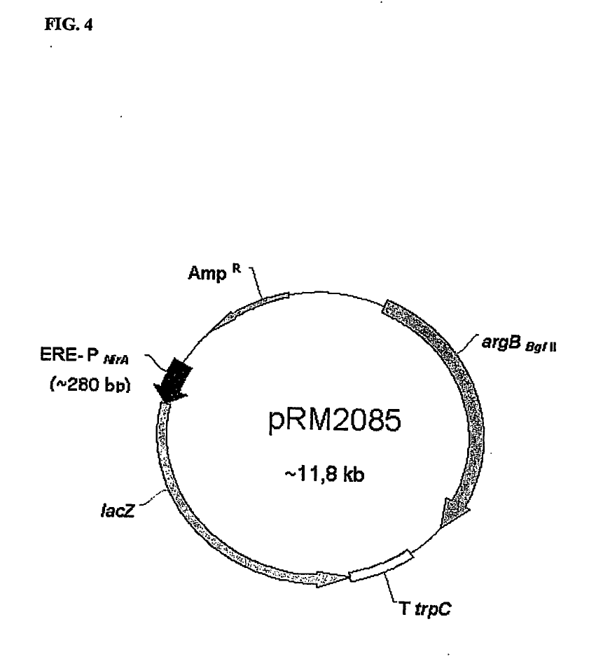

Construction of Plasmid pRM2085 (FIG. 4)

3xERE sequence followed by a 117 bp fragment of the URA3 promoter of S. cerevisiae (Acc. M12926, bases 89-205) (Klein-Hitpass et al., 1988) and a 94 bp fragment of the nirA promoter of A. nidulans (Acc. M68900) was amplified by using PCR and was cloned as a BamHI/BglII fragment into the BamHI site of pAN923-42_{BglII} (Van Gorcom et al. 1986). The structural gene is the reporter lacZ from E. coli (Acc. V00296). The marker gene is argB form A. nidulans (Acc. AB020737). Terminator sequences include the trpC terminator from Aspergillus nidulans (Acc. X02390).

Construction of pERE URA JUNK

The nirA promoter fragment of pRM2085 was discarded and replaced by a 94 bp fragment of the ORF of the amp^{R} gene of pBSK+ (Stratagene, La Jolla, Calif., USA).

Construction of pERE JUNK nirA

The URA3 promoter fragment of pRM2085 was discarded and replaced by a 117 bp fragment of the ORF of the amp^{R} gene of pBSK+ (Stratagene, La Jolla, Calif., USA).

Construction of pRM alcA

The ERE URA nirA cassette of pRM2085 was discarded and replaced by a 427 bp fragment of the alcA promoter of A. nidulans (Gwynne, D. I, et al., Gene 51, 205-216 (1987)).

### EXAMPLE 2

### Culture Conditions

Aspergillus strains were grown for 12-14 hours at 37° C. at 180 rpm in minimal media (Pontecorvo et al. 1953) with appropriate supplements. To test the different inducer concentrations the strains were harvested by filtration and aliquots were transferred to fresh media containing the different inducer concentrations and additionally grown for 8 hours. At the end mycelium was harvested by filtration and frozen in liquid nitrogen. For time curve experiments the strains were harvested and additionally grown for 24 hours and after 2, 4, 6, 8 and 24 hours samples were taken and frozen in liquid nitrogen. FIG. 9 shows the expression levels of the reporter constructs after activation with inducers, either with ethanol (alc) or with DES (hER). An equal amount of fungal cell mass has been transferred to fresh medium and aliquots have been subjected to induction by the agonists. After sampling at desired time points the wet cell pellet has been processed as described.

### EXAMPLE 3

### Reporter Enzyme Assay

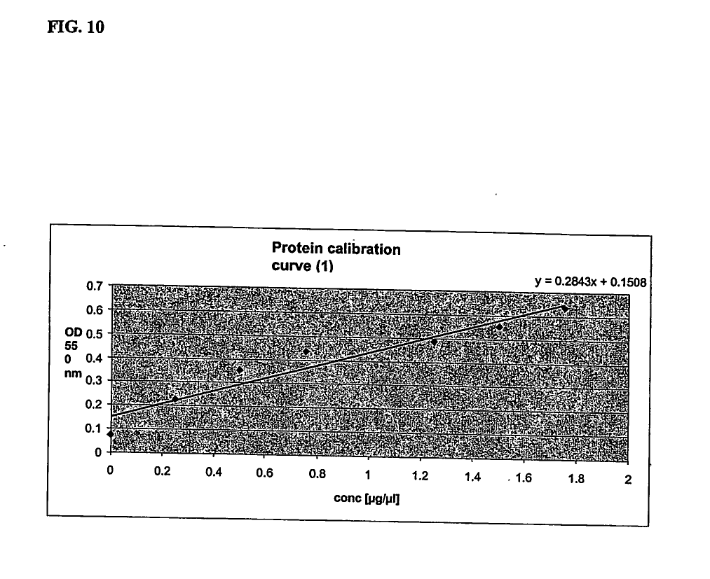

NaPO₄ buffer and glasbeads (0.75-1.0 mm) were added to the frozen mycelia and cells were destroyed by the use of the Hybaid RiboLyser. Cell debris was separated by centrifugation and the supernatant was used for enzyme assays Protein concentration was determined using the BCA assay of Pierce and the specific β-galactosidase activity was determined by the use of the protocol of Invitrogen. FIG. 10 shows a protein standard curve obtained with bovine serum albumin (BSA) (Table 1) under the reaction conditions used throughout the whole set of experiments. The determination of protein content of a given sample was carried out according to these standard conditions and specific enzymatic activity levels (units β-galactosidase) have been correlated to units per milligram protein (units per mg protein).

A calibration curve was plotted showing the relationship between the concentration of DES [μg/μl] and the protein concentration [units/mg]. The protein concentration of the samples was determined to express the specific activity as units per milligram protein (Table 2).

TABLE 1
Standard curve with BSA

| Conc | |
|---|---|
| [μg/μl] | OD 550 nm |
| 0 | 0.073 |
| 0.25 | 0.226 |
| 0.5 | 0.354 |
| 0.75 | 0.435 |
| 1.25 | 0.484 |
| 1.5 | 0.554 |
| 1.75 | 0.636 |

TABLE 2
Amount of protein present in the sample (mg/ml)

| | | | |
|---|---|---|---|
| OD 550 | conc | dilution | concentration |

pRM2085

| | | | | |
|---|---|---|---|---|
| 0 DES | 0.476 | 1.14386173 | 10 | 11.4386173 |
| 1 pM DES | 0.367 | 0.76046404 | 10 | 7.60464042 |
| 10 pM DES | 0.372 | 0.77805109 | 10 | 7.78051092 |
| 100 pM DES | 0.344 | 0.67956361 | 10 | 6.79563612 |
| 1 nM DES | 0.314 | 0.57404131 | 10 | 5.74041312 |
| 10 nM DES | 0.282 | 0.46148419 | 10 | 4.61484192 |
| 100 nM DES | 0.299 | 0.52128016 | 10 | 5.21280162 |

pRM2119

| | | | | |
|---|---|---|---|---|
| 0 DES | 0.335 | 0.64790692 | 10 | 6.47906922 |
| 1 pM DES | 0.423 | 0.957439 | 10 | 9.57439002 |
| 10 pM DES | 0.395 | 0.85895152 | 10 | 8.58951522 |
| 100 pM DES | 0.481 | 1.16144878 | 10 | 11.6144878 |
| 1 nM DES | 0.353 | 0.7112203 | 10 | 7.11220302 |
| 10 nM DES | 0.365 | 0.75342922 | 10 | 7.53429222 |
| 100 nM DES | 0.338 | 0.65845915 | 10 | 6.58459152 |

pRM2124

| | | | | |
|---|---|---|---|---|
| 0 DES | 0.398 | 0.86950375 | 10 | 8.69503752 |
| 1 pM DES | 0.318 | 0.58811095 | 10 | 5.88110952 |
| 10 pM DES | 0.359 | 0.73232476 | 10 | 7.32324762 |
| 100 pM DES | 0.433 | 0.9926131 | 10 | 9.92613102 |
| 1 nM DES | 0.319 | 0.59162836 | 10 | 5.91628362 |
| 10 nM DES | 0.25 | 0.34892707 | 10 | 3.48927072 |
| 100 nM DES | 0.304 | 0.53886721 | 10 | 5.38867212 |

### EXAMPLE 4

### Growth Test/X Gal Activity Test on Plates at Different Ligand Concentrations

Induction of estrogen receptor expression via the addition of 0.1 pM, 1 pM, 10 pM, 100 nM, and 1 μM of DES inducer to media plates harboring the Xgal substrate for LacZ in the transformed strain: A. nidulans argB2, riboA1, pyrG 89, pyroA4. FIG. 6. Results show that DES concentrations up to 20 μM do not affect the growth of a wild type strain. Activation of the reporter construct at 1 nM DES was shown in the transformed fungi. Growth inhibition of strains transformed with the hER can be seen at 1 nM DES while strong growth inhibition was exhibited at 100 nM DES in strains transformed with reporter constructs.

### EXAMPLE 5

### X Gal Activity Test in Liquid Culture

FIG. 7 shows the assay for β-Gal activity in liquid culture containing Xgal. One unit is defined as the amount of enzyme that will hydrolyze 1 nM of ONPG per 5 minute at 28° C. The results indicate that the inducer DES (at 1 nM and 10 mM) strongly induces the expression of LacZ based upon β-Gal activity. See Example 3.

### EXAMPLE 6

### β-gal Activity in pRM Constructs

β-gal activity using different concentrations of DES was examined. Table 3 shows the induction levels using different pRM reporter constructs. FIG. 8 depicts the assay of Table 3 in a graphical representation.

TABLE 3

| | pRM2085 | pRM2119 | pRM2124 |
|---|---|---|---|
| 0 DES | 55.859122 | 59.5322463 | 55.7568174 |
| 1 pM DES | 84.8681882 | 31.6156096 | 54.9130459 |
| 10 pM DES | 77.8128134 | 45.2067375 | 90.1990494 |
| 100 pM DES | 553.769052 | 82.2380063 | 225.614743 |
| 1 nM DES | 1822.68374 | 225.026402 | 777.368521 |
| 10 nM DES | 2511.75733 | 248.157062 | 1448.33221 |
| 100 nM DES | 2476.80067 | 278.163776 | 1010.09359 |

### EXAMPLE 7

### β-gal Activity in pERE Constructs

β-gal activity in each pERE constructs were assayed after induction with 1 nM DES. The alcA expression was also assayed for comparison. See Example 3. The combination pERE URA Junk results in the highest induction level. Table 4 shows the induction of levels β-gal in pERE constructs over time.

TABLE 4

| | pERE | pERE | | pRM |
|---|---|---|---|---|
| Time | URA NirA | URA JUNK | pERE JUNK NirA | alcA |
| 0 | 6.77 | 130.39 | 5.22 | 374.70 |
| 2 | 178.42 | 2377.83 | 185.49 | 506.66 |
| 4 | 427.27 | 5214.17 | 336.55 | 714.43 |
| 6 | 753.22 | 7064.16 | 460.60 | 1327.44 |
| 8 | 928.45 | 7699.50 | 533.93 | 1431.02 |
| 24 | 1231.44 | 7884.99 | 771.55 | 6082.59 |
| Inducibility | 182.02 | 60.47 | 147.84 | 16.23 |

### EXAMPLE 8

### β Gal Activity in pERE URA nirA Constructs Using Different Carbon and Nitrogen Sources

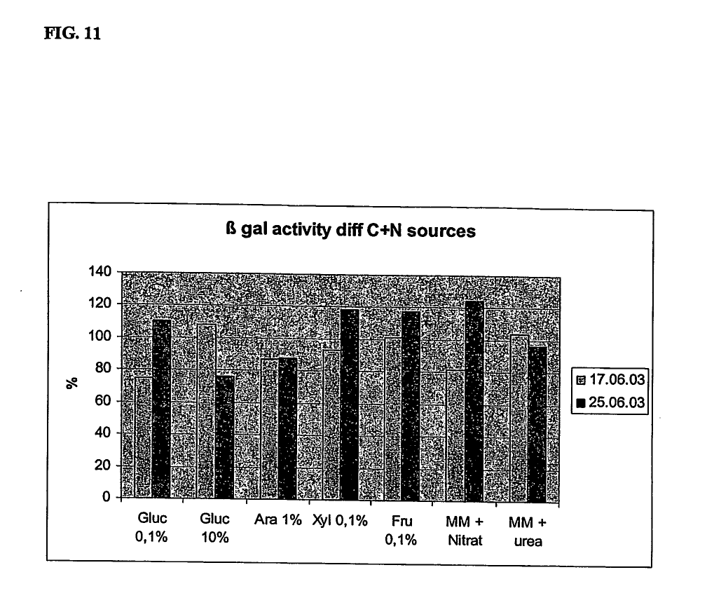

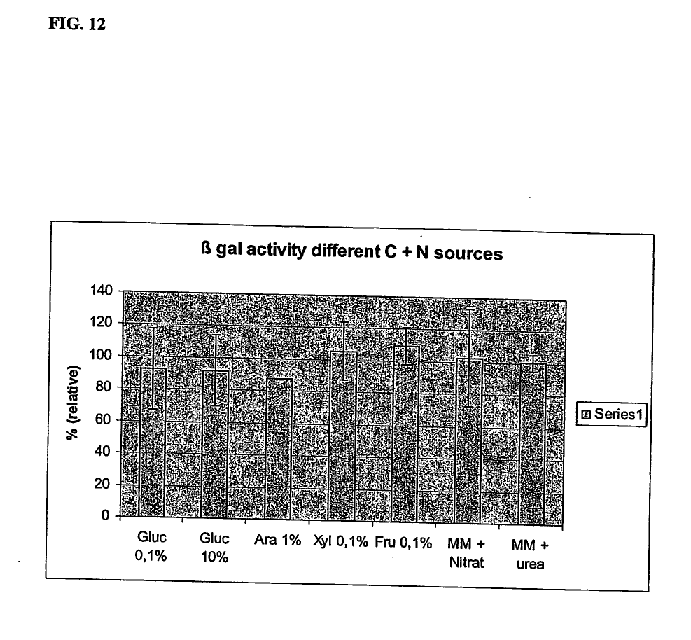

The strain was grown under standard conditions using the defined Aspergillus minimal medium with different carbon and nitrogen sources. The cultures were harvested after 8 hours of induction with 1 nM DES and the specific beta-galactosidase activities were measured and expressed as units per mg protein. As a standard, the cultures grown on 1% glucose as carbon source and 5 mM ammonia as nitrogen source were collected and analyzed and the average units per mg protein of these triplicate samples were used to set as the reference expression level, i.e. 100%. Tables 5 and 6 (FIGS. 11 and 12 are graphical representations of Tables 5 and 6, respectively) show percentage of the reference level obtained from cultures grown on different concentrations and sources of carbon and nitrogen. (Gluc., glucose; Ara., arabinose; Xyl., xylose; MM., minimal medium).

TABLE 5

| | % 17.06.03 | % 25.06.03 |
|---|---|---|
| Gluc 0.1% | 74.7775982 | 110.017095 |
| Gluc 10% | 107.7721337 | 76.3113009 |
| Ara 1% | 87.27744948 | 87.7083759 |
| Xyl 0.1% | 92.97056097 | 118.357072 |
| Fru 0.1% | 101.5312708 | 117.810406 |
| MM + Nitrat | 81.44317061 | 124.078639 |
| MM + urea | 104.050649 | 96.7442253 |

TABLE 6

| | |
|---|---|
| Gluc 0.1% | 92.3973468 |
| Gluc 10% | 92.0417173 |
| Ara 1% | 87.49291271 |
| Xyl 0.1% | 105.6638165 |
| Fru 0.1% | 109.6708384 |
| MM + Nitrat | 102.7609047 |
| MM + urea | 100.3974371 |

### REFERENCES

Klein-Hitpass L, Ryffel G U, Heitlinger E, Cato A C. A 13 bp palindrome is a functional estrogen responsive element and interacts specifically with estrogen receptor. Nucleic Acids Res. 1988 Jan. 25; 16(2):647-63
Pierrat B, Heery D M, Lemoine Y, Losson R: Functional analysis of the human estrogen receptor using a phenotypic transactivation assay in yeast. Gene. 1992 Oct. 1; 119(2):23745
Pontecorvo G, Roper J A, Hemmons L M, MacDonald K D, and Bufton A W J.: The genetics of Aspergillus nidulans. Adv Genet 5 (1953): 141-238
Van Gorcom, R. F. M., Punt P J, Pouwles P H and van den Hondel CAMJJ: A system for the analysis of expression signals in Aspergillus. Gene 48 (1986) 211-217

What is claimed is:

1. A steroid inducible promoter expression system in a filamentous fungus or a unicellular yeast, wherein the inducible promoter expression system can be modulated.

2. A steroid inducible promoter expression system in a filamentous fungus or a unicellular yeast comprising a regulated gene expression cassette which comprises:
(a) a first promoter operatively linked to a regulator protein; and
(b) a regulatable promoter operatively linked to a target nucleic acid molecule, the regulatable promoter being activated by the regulator protein in the presence of an exogenous inducer, wherein addition of the exogenous inducer activates expression of the target nucleic acid molecule and wherein withdrawal of the exogenous inducer or its activity inactivates expression of the target nucleic acid molecule.

3. The steroid inducible promoter expression system of claim 2, wherein the regulator protein is expressed from a nucleic acid construct that is not physically linked to the regulatable promoter.

4. The steroid inducible promoter expression system of claim 2, wherein the regulatable promoter is an estrogen receptor promoter.

5. The steroid inducible promoter expression system of claim 1 or 2 wherein the promoter further comprises a stuffer fragment for activation or repression.

6. A vector selected from the group consisting of pRM2085, pRM2124, pRM2119, pERE URA JUNK, pERE URA nirA and pERE JUNK nirA expressed in a filamentous fungus or a methylotrophic yeast.

7. The steroid inducible promoter expression system of claim 1 or 2, wherein the filamentous fungus is selected from the group consisting of A. nidulans, A. niger, A. oryzae, A. awamori, A. chrysogenum, A. saitoi, A. tubigensis, Trichoderma sp., Chrysosporium lucknowense, Fusarium sp., Mucor sp., Ashbya gossipii, Penicillium sp., and Neurospora crassa.

8.-13. (canceled)

14. A steroid inducible promoter expression system of claim 1 or 2, wherein the unicellular yeast is selected from the group consisting of Pichia sp., Hansenula sp., Kluyveromyces sp., Candida sp. and Torulopsis sp.

15. A method for modulating gene expression in a filamentous fungus or a unicellular yeast comprising inducing a target nucleic acid molecule using a regulated gene expression cassette which comprises:
(a) a first promoter operatively linked to a regulator protein; and
(b) a regulatable promoter operatively linked to a target nucleic acid molecule, the regulatable promoter being activated by the regulator protein in the presence of an exogenous inducer, wherein addition of the exogenous inducer activates expression of the target nucleic acid molecule and wherein withdrawal of the exogenous inducer or its activity inactivates expression.

16. A filamentous fungus or a unicellular yeast comprising a steroid inducible expression system that can be modulated.

17. The filamentous fungus or unicellular yeast of claim 16, wherein a target nucleic acid molecule is modulated by a regulatable gene expression cassette which comprising:
(a) a first promoter operatively linked to a regulator protein; and
(b) a regulatable promoter operatively linked to a target nucleic acid molecule, the regulatable promoter being activated by the regulator protein in the presence of an exogenous inducer, wherein addition of the exogenous inducer activates expression of the target nucleic acid molecule wherein withdrawal of the exogenous inducer or its activity inactivates expression.

18. The filamentous fungus of claim 16 or 17, wherein the filamentous fungus is selected from the group consisting of A. nidulans, A. niger, A. oryzae, A. awamori, A. chrysogenum, A. saitoi, A. tubigensis, Trichoderma sp., Chrysosporium lucknowense, Fusarium sp., Mucor sp., Ashbya gossipii, Penicillium sp., and Neurospora crassa.

19. The unicellular yeast of claim 16 or 17 wherein the unicellular yeast is selected from the group consisting of Pichia sp., Hansenula sp., Kluyveromyces sp., Candida sp. and Torulopsis sp.

20. The steroid inducible promoter expression system of claim 7 wherein the Trichoderma sp. is selected from the group consisting of T. reesei, T. viridae and T. harzianum.

21. The steroid inducible promoter expression system of claim 7 wherein the Fusarium sp. is selected from the group consisting of Fusarium gramineum and Fusarium venenatum.

22. The steroid inducible promoter expression system of claim 14 wherein the Pichia sp. is selected from the group consisting of Pichia pastoris, Pichia finlandica, Pichia trehalophila, Pichia koclamae, Pichia membranaefaciens, Pichia minuta, Ogataea minuta, Pichia lindneri, Pichia opuntiae, Pichia thermotolerans, Pichia salictaria, Pichia guercuum, Pichia pijperi, Pichia stiptis and Pichia methanolica.

23. The steroid inducible promoter expression system of claim 14 wherein the Hansenula sp. is Hansenula polymorpha.

24. The steroid inducible promoter expression system of claim 14 wherein the Kluyveromyces sp. is Kluyveromyces lactis.

25. The steroid inducible promoter expression system of claim 14 wherein the Candida sp. is Candida albicans.

26. The filamentous fungus or unicellular yeast of claim 18 wherein the Trichoderma sp. is selected from the group consisting of T. reesei, T. viridae and T. harzianum.

27. The filamentous fungus or unicellular yeast of claim 18 wherein the Fusarium sp. is selected from the group consisting of Fusarium gramineum and Fusarium venenatum.

28. The filamentous fungus or a unicellular yeast of claim 19 wherein the Pichia sp. is selected from the group consisting of Pichia pastoris, Pichia finlandica, Pichia trehalophila, Pichia koclamae, Pichia membranaefaciens, Pichia minuta, Ogataea minuta, Pichia lindneri, Pichia opuntiae, Pichia thermotolerans, Pichia salictaria, Pichia guercuum, Pichia pijperi, Pichia stiptis and Pichia methanolica.

29. The filamentous fungus or unicellular yeast of claim 19 wherein the Hansenula sp. is Hansenula polymorpha.

30. The filamentous fungus or unicellular yeast of claim 19 wherein the Kluyveromyces sp. is Kluyveromyces lactis.

31. The filamentous fungus or unicellular yeast of claim 19 wherein the Candida sp. is Candida albicans.
